(19) United States
(12) Patent Application Publication
(10) Pub. No.: US 2008/0071706 A1
(43) Pub. Date: Mar. 20, 2008

(54) Method of designing high-affinity peptide, method of preparing high-affinity peptides, computer-readable storage medium storing a program for designing high-affinity peptide, apparatus for designing high-affinity peptide, and high-affinity peptide

(75) Inventors: Hiroyuki Honda, Nagoya (JP); Mina Okochi, Nagoya-shi (JP); Chiaki Kaga, Nagoya-shi (JP); Yasuyuki Tomita, Nagoya-shi (JP); Ryuji Kato, Nagoya-shi (JP)

Correspondence Address: EDWARDS ANGELL PALMER & DODGE LLP, P.O. BOX 55874, BOSTON, MA, 02205, US

(73) Assignee: National University Corporation Nagoya University, Nagoya-shi (JP)

(21) Appl. No.: 11/882,512

(22) Filed: Aug. 2, 2007

(30) Foreign Application Priority Data
Sep. 8, 2006 (JP) 2006-243883

(51) Int. Cl. G06F 15/18; C07K 5/00; G06N 7/02

(52) U.S. Cl. 706/2; 530/330; 706/52

(57) ABSTRACT

It is an object of the present invention to provide means for efficiently obtaining peptides that exhibit a high affinity toward a given target. Peptides are designed by the steps of (1) performing an affinity assay using a plurality of peptides having different peptide sequences and a target to obtain affinity data for each of the peptide sequences toward the target; (2) selecting high-affinity peptide sequences and low-affinity peptide sequences; (3) digitizing predetermined property(ies) of amino acids for each location from N-terminal or C-terminal to transform each of the selected peptide sequences into numerical data; (4) performing Fuzzy Neural Network analysis by using the obtained numerical data as input variables to construct a prediction model; (5) extracting from the constructed prediction model one or two or more rules wherein the amino acids and the properties are related to each other on one or more locations on the sequences, said rules representing the characteristics of high-affinity peptide sequence; and (6) designing a peptide according to the extracted rules.

### CROSS REFERENCE TO RELATED APPLICATIONS

This application claims priority to Japanese application No. 2006-243883, filed Sep. 8, 2006, which is hereby incorporated by reference in their entirety.

### FIELD OF THE INVENTION

This invention relates to a method for designing peptides with a high affinity toward a given target. This invention also provides a method for preparing high-affinity peptides using the designing method. This invention also provides a computer-readable storage medium storing a program for designing high-affinity peptides and an apparatus for designing high-affinity peptides. In addition, the present invention provides a peptide exhibiting a high affinity toward a given target and comprising a specific peptide sequence.

### BACKGROUND OF THE INVENTION

Amongst numerous bioactivities of peptides is an induction of cell adhesion. Adhesion peptides having a cell adhesion induction activity have been focused on from aspects of cell targeting and development of serum-free culture. A variety of adhesion peptides have been explored to date, including a cell adhesion peptide Arg-Gly-Asp (RGD sequence), a representative adhesion peptide which has been searched out in the sequence of fibronectin present in extracellular matrix (Pierschbacher, M. D. & Rouslahti, E. (1984) Nature 309, 30-33, 1984) and is known to be recognized by integrin. RGD sequence have been focused on as a medium for achieving cell targeting and efficient cell culture, and studied for its potential use in the field of regenerative medicine. Synthetically produced peptides can be deemed as highly safe adhesion materials with expected applicability in medicines.

In the recent years, drug delivery system (DDS) “for selectively delivering a required amount of drugs to a specific lesion requiring the drug at a specific time the drug is needed” has been intensively studied, especially for developing a drug targeting technology. Cancer cells typically have cell-specific receptors on their surfaces. Various ligand molecules targeting these receptors, and monoclonal antibodies targeting any antigenic substances on cell surfaces have been developed for use as targeting molecule or carrier. Therapies using such a DDS have been considered prospective and drawing a keen interest since they provide means for imaging a target tissue and for selectively transporting a drug to target cells. Because of their ready synthesis and chemical linkage with drugs as well as flexibility in designing them, peptides are also highly expected carrier for DDS. The research group including the present inventors have provided numerous reports concerning peptides: (Kato, R., Okuno, Y., Kaga, C., Kunimatsu, M., Kobayashi, T. and Honda, H. (2006) J. Peptide Res. 66(suppl. 1), 146-153.; Kato, R. Kaga, C., Kunimatsu, M., Kobayashi, T. and Honda, H. (2006) J. Biosci. Bioeng. 101, 485-495.; Okochi, M., Nakanishi, M., Kato, R., Kobayashi, T. and Honda, H. (2006) FEBS Lett. 580, 885-889.), knowledge relating to protein engineerings utilizing Informatics (Kato, R., Nakano, H., Konishi, H., Kato, K., Koga, Y., Yamane, T., Kobayashi, T. and Honda, H. (2005) J. Mol. Biol. 351, 683-692.).

### SUMMARY OF THE INVENTION

Thus, peptides have been widely considered as prospective target in a variety of fields, and development or creation of peptides which exhibit a high affinity (high adhesiveness) toward a given target are in great demand. However, peptides in general are composed of a combination of amino acids (a combination of essentially 20 amino acids for natural peptides), leading to a tremendous number of possible combinations resulting in peptide sequences. It is thus extremely difficult to find out a peptide of interest.

It is thus an object of the present invention to provide a means for efficiently obtaining peptides exhibiting a high affinity toward a given target. It is also an object of the present invention to provide peptides (high-affinity peptides) that exhibit a high affinity toward a target and are expected to be usable as adhesion peptides in a variety of fields.

In order to attain the object, the present inventors sought to establish a technique for designing high-affinity peptides with a high through-put. Specifically, Fuzzy Neural Network or FNN, an information processing and analysis tool, was used in analyzing experimental data of affinity assays, thus constructing a technique a concept of which is shown in FIG. 1. A series of experiments were then performed as follows to demonstrate the validity of the technique. First, arrays of random peptide sequences of 4-mer were synthesized on peptide arrays, and an affinity assay using cells was performed to screen high-affinity peptide and low-affinity peptide sequences. Subsequently, physical properties (such as size, hydrophobicity, and charges) of amino acids constituting the selected peptide sequences are digitized for each location on the sequences to yield peptide sequence data for FNN analysis. As a result, a correlation between the peptide sequences (properties of each amino acid residue) and affinity was successfully elucidated as rules. Peptides that were newly synthesized according to the rules exhibited a high affinity (adhesion activity) under a serum-free condition. In addition, it was found that repetitive affinity assays and FNN analyses yield rules with a greater validity. Thus, it was demonstrated that the instant technique is a valid tool for successfully obtaining peptides with a high affinity.

The present invention is primarily based on the above findings and results and provides a method for designing high-affinity peptides and the like as follows.

[1] A method for designing a high-affinity peptide, comprising the steps of (1) to (6) of:

(1) performing an affinity assay using a plurality of peptides having different peptide sequences and a target to obtain affinity data for each of the peptide sequences toward the target;

(2) selecting high-affinity peptide sequences and low-affinity peptide sequences;

(3) digitizing predetermined properties of amino acids for each location from N-terminal or C-terminal to transform each of the selected peptide sequences into numerical data;

(4) performing Fuzzy Neural Network analysis by using the obtained numerical data as input variables to construct a prediction model;

(5) extracting from the constructed prediction model one or two or more rules wherein the amino acids and the properties are related to each other on one or more locations on the sequences, said rules representing the characteristics of high-affinity peptide sequence;

(6) designing a peptide according to the extracted rules.

[2] The method according to [1], wherein two or more rules are extracted in the step of (6).

[3] The designing method according to [1] or [2], wherein a plurality of peptides having different peptide sequences are designed in the step of (6), further comprising the step of (7) of performing the steps of (1) to (6) using the designed plurality of peptides.

[4] A designing method according to [3], wherein the step of (7) is repeated two or more times.

[5] The designing method according to any one of [1] to [4], wherein said affinity assay is performed using a peptide chip comprising a plurality of peptides segmented according to each peptide sequence and immobilized on a substrate.

[6] The designing method according to any one of [1] to [5], wherein the plurality of peptides in the step of (1) are equal in length.

[7] The designing method according to any one of [1] to [6], wherein the plurality of peptides in the steps of (1) comprises 3 to 15 amino acids.

[8] The designing method according to any one of [1] to [7], wherein the plurality of peptides in the step of (1) comprises a set of peptides having randomly selected amino acid sequences.

[9] The designing method according to any one of [1] to [8], wherein said target is biopolymer such as cell, protein or peptide, or particulate or base made of metal, semiconductor, inorganic material or synthetic polymer.

[10] The designing method according to any one of [1] to [9], wherein the properties in the step of (3) are one or more properties selected from the group consisting of size, hydrophobicity, charges, isoelectric point, presence or absence of branch, presence or absence of sulfur element, presence or absence of hydroxyl, presence or absence of benzene ring, and presence or absence of heterocycle.

[11] The designing method according to [10], wherein the properties are two or more properties selected from the above group.

[12] The designing method according to any one of [1] to [9], wherein the properties in the step of (3) are size, hydrophobicity and charges.

[13] A method of preparing a high-affinity peptide, comprising preparing a peptide designed by the designing method according to any one of [1] to [12].

[14] Computer-readable storage medium storing a program for performing the following steps to design a high-affinity peptide:

digitizing predetermined properties of amino acids for each location from N-terminal or C-terminal to transform into numerical data a plurality of peptide sequences having different affinities toward a given target;

performing Fuzzy Neural Network analysis by using the obtained numerical data as input variables to construct a prediction model; and

extracting from the constructed prediction model one or two or more rules wherein the amino acids and the properties are related to each other on one or more locations on the sequences, said rules representing the characteristics of high-affinity peptide sequence.

[15] Computer-readable storage medium storing a program for performing the following steps to design a high-affinity peptide:

selecting high-affinity peptide sequences and low-affinity peptide sequences based on affinity data for each of the peptide sequences toward the target, obtained through affinity assay using a plurality of peptides having different sequences and a target;

digitizing predetermined properties of amino acids for each location from N-terminal or C-terminal to transform into numerical data the high-affinity peptide sequences and low-affinity peptide sequences;

performing Fuzzy Neural Network analysis by using the obtained numerical data as input variables to construct a prediction model; and

extracting from the constructed prediction model rules wherein the amino acids and the properties are related to each other on one or more locations on the sequences, said rules representing the characteristics of high-affinity peptide sequence.

[16] Computer-readable storage medium storing a program for performing the following steps to design high-affinity peptides:

digitizing predetermined properties of amino acids for each location from N-terminal or C-terminal to transform into numerical data a plurality of peptide sequences having different affinities toward a given target;

performing Fuzzy Neural Network analysis by using the obtained numerical data as input variables to construct a prediction model;

extracting from the constructed prediction model one or two or more rules wherein the amino acids and the properties are related to each other on one or more locations on the sequences, said rules representing the characteristics of high-affinity peptide sequence; and

designing a peptide according to said rule.

[17] Computer-readable storage medium storing a program for performing the following steps to design high-affinity peptides:

selecting high-affinity peptide sequences and low-affinity peptide sequences based on affinity data for each of the peptide sequences toward the target, obtained through affinity assay using a plurality of peptides having different sequences and a target;

digitizing predetermined properties of amino acids for each location from N-terminal or C-terminal to transform into numerical data the high-affinity peptide sequences and low-affinity peptide sequences;

performing Fuzzy Neural Network analysis by using the obtained numerical data as input variables to construct a prediction model;

extracting from the constructed prediction model one or two or more rules wherein the amino acids and the properties are related to each other on one or more locations on the sequences, said rules representing the characteristics of high-affinity peptide sequence; and

designing a peptide according to said rules.

[18] An apparatus for designing a high-affinity peptide comprising:

means for digitizing predetermined properties of amino acids for each location from N-terminal or C-terminal to transform into numerical data a plurality of peptide sequences having different affinities toward a given target;

means for performing Fuzzy Neural Network analysis by using the obtained numerical data as input variables to construct a prediction model; and

means for extracting from the constructed prediction model one or two or more rules wherein the amino acids and the properties are related to each other on one or more locations on the sequences, said rules representing the characteristics of high-affinity peptide sequence.

[19] An apparatus for designing a high-affinity peptide comprising:

means for selecting high-affinity peptide sequences and low-affinity peptide sequences based on affinity data for each of the peptide sequences to ward the target, obtained through affinity assay using a plurality of peptides having different sequences and a target;

means for digitizing predetermined properties of amino acids for each location from N-terminal or C-terminal to transform into numerical data the high-affinity peptide sequences and low-affinity peptide sequences;

means for performing Fuzzy Neural Network analysis by using the obtained numerical data as input variables to construct a prediction model; and

means for extracting from the constructed prediction model one or two or more rules wherein the amino acids and the properties are related to each other on one or more locations on the sequences, said rules representing the characteristics of high-affinity peptide sequence.

[20] An apparatus for designing a high-affinity peptide comprising:

means for digitizing predetermined properties of amino acids for each location from N-terminal or C-terminal to transform into numerical data a plurality of peptide sequences having different affinities toward a given target;

means for performing Fuzzy Neural Network analysis by using the obtained numerical data as input variables to construct a prediction model; and

means for extracting from the constructed prediction model one or two or more rules wherein the amino acids and the properties are related to each other on one or more locations on the sequences, said rules representing the characteristics of high-affinity peptide sequence; and

means for designing a peptide according to said rules.

[21] An apparatus for designing a high-affinity peptide comprising:

means for selecting high-affinity peptide sequences and low-affinity peptide sequences based on affinity data for each of the peptide sequences to ward the target, obtained through affinity assay using a plurality of peptides having different sequences and a target;

means for digitizing predetermined properties of amino acids for each location from N-terminal or C-terminal to transform into numerical data the high-affinity peptide sequences and low-affinity peptide sequences;

means for performing Fuzzy Neural Network analysis by using the obtained numerical data as input variables to construct a prediction model;

means for extracting from the constructed prediction model one or two or more rules wherein the amino acids and the properties are related to each other on one or more locations on the sequences, said rules representing the characteristics of high-affinity peptide sequence; and

means for designing peptides according to said rules.

[22] A high-affinity peptide comprising an amino acid sequence shown in any one of SEQ ID NOs: 1-70.

According to the present invention, it is possible to design, prepare or manufacture peptides with a high affinity toward a given target in a high through-put. In addition, a target of interest can be a variety of substance. Accordingly, present invention allows for designing, preparing or manufacturing a peptide with a high affinity toward a variety of targets. Thus, the present invention also affords universality.

### BRIEF DESCRIPTION OF THE DRAWINGS

These and other objectives and technical advantages of the present invention will be readily apparent from the following description of the preferred exemplary embodiments of the invention in conjunction with the accompanying drawings, in which:

FIG. 1 depicts a strategy of designing technique of peptides exhibiting a high affinity.

FIG. 2 depicts indices of size, hydrophobicity and charges of amino acids.

FIG. 3 depicts a structure of ANN.

FIG. 4 depicts a structure of FNN.

FIG. 5 depicts a sigmoid function.

FIG. 6 depicts a schematic view of FNN in two-input, 2 branches and one output format.

FIG. 7 depicts an example of Fuzzy rule. “S” in the table represents “small”, and “B” represents “big”, respectively.

FIG. 8 depicts a sequence rule table generated by the first FNN analysis.

FIG. 9 depicts a table summarizing extracted peptide-designing rules.

FIG. 10 depicts a sequence rule table generated by the second FNN analysis (using a peptide according to Rule (I)).

FIG. 11 depicts a sequence rule table generated by the second FNN analysis (using a peptide according to Rule (II)).

FIG. 12 depicts a graph showing results of cell adhesion assary. Graph (a) shows a comparison of the test results between rules (I) to (V). Ordinate axis represents average values of relative fluorescence intensity. * denotes that the value of interest is statistically significant in comparison to a control (p<0.05). Graph (b) shows a comparison of the test results between rules (IV) and (V). Columns with slash therein represent test results of reverse rules. Ordinate axis represents average values of relative fluorescence intensity. ** denotes that the value of interest is statistically significant in comparison to the reverse rules.

FIG. 13 depicts a table of peptides synthesized according to the extracted rules (IV), sorted out in the order of affinity, from the one with a greatest affinity at the top, to the one with a least affinity on the bottom.

FIG. 14 depicts a table of peptides synthesized according to the extracted rules (V), sorted out in the order of affinity, from the one with a greatest affinity at the top, to the one with a least affinity on the bottom.

FIG. 15 depicts an example of hardware structure of the designing apparatus according to the present invention, which comprises a main controller 10, a main memory unit 20, a temporary memory unit 30, an input/output controller 40, an input unit 50 and an output unit 60.

FIG. 16 depicts a flow chart showing a processing procedure by the designing apparatus according to the present invention.

FIG. 17 depicts a flow chart showing a processing procedure by software for constructing a prediction model by FNN analysis.

FIG. 18 depicts a flowchart showing a processing procedure by another designing apparatus according to the present invention.

FIG. 19 depicts a flowchart showing a processing procedure by another designing apparatus according to the present invention.

FIG. 20 depicts a flowchart showing a processing procedure by another designing apparatus according to the present invention.

### DETAILED DESCRIPTION OF THE INVENTION

### 1. Method for Designing High-Affinity Peptide

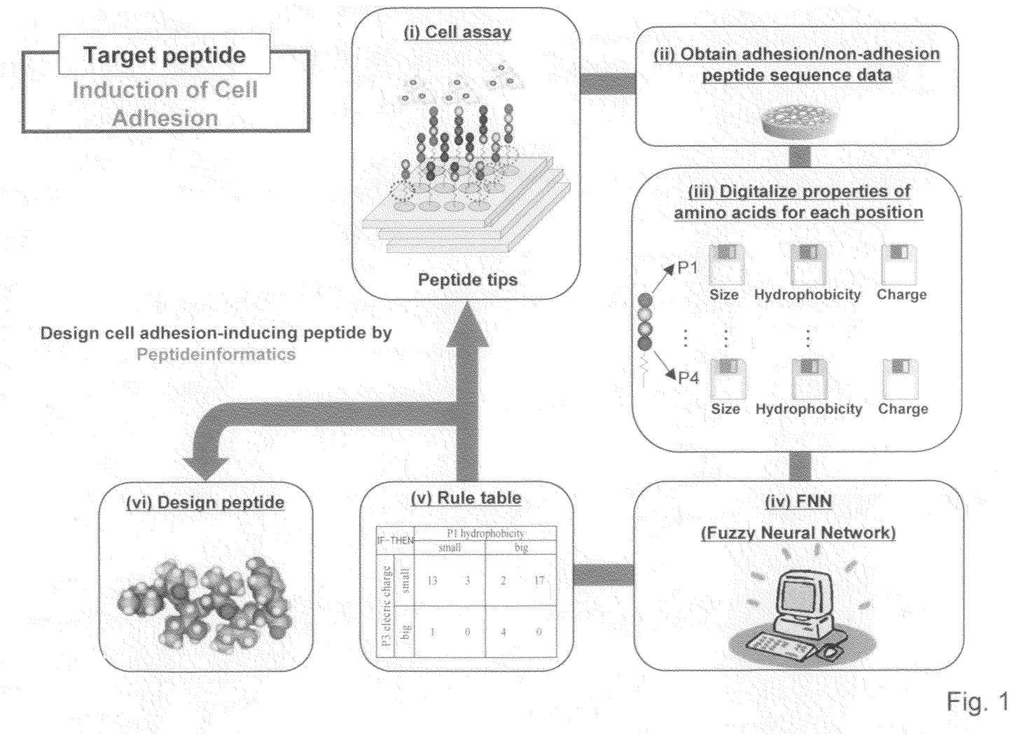

A designing method according to the present invention will be now described with reference to FIG. 1 which schematically depicts a procedure of the designing method according to the present invention.

### (1) Obtain Affinity Data (FIGS. 1 (i), and (ii))

In the first step of the designing method according to the present invention, an affinity assay is performed using a plurality of peptides having different peptide sequences (hereinafter also referred to as “sample peptide”) and a target to obtain affinity data for each of the peptides toward the target (step (1)).

The number of sample peptides is not particularly limited. However, a variety or peptides are preferably used for efficient designing of peptides. Nevertheless, since an increased number of sample peptides used will add to complexity in the following analysis, the number of peptides will be in the range of 10 to 5000, or preferably in the range of 100 to 1000.

Peptides having different peptide sequences, i.e. plural types of peptides are used in the affinity assay. Therefore, as long as plural types of peptides are used, the specific sequence of each of the peptides is not limited. However, any bias in the peptide sequences should be eliminated in order to extract more reliable rules in the following FNN analysis. Thus, a set of peptides having randomly selected amino acid sequences (a peptide library having random sequences) is preferably used. Typically, a plurality of peptides for each of the types is used as in the case of protein chip used in following example.

Typically, sample peptides are composed of those amino acids constituting proteins in vivo, i.e. alanine, arginine, asparagine, asparaginic acid, cysteine, glutamic acid, glutamine, glycine, histidine, isoleucine, leucine, lysine, methionine, phenylalanine, proline, serine, threonine, tryptophane, tyrosine, valine etc. However, any amino acids that can constitute a peptide can be used as a component amino acid of the sample peptide according to the present invention. For example, in addition to L-amino acids, D-amino acids, modified (acetylated, methylated, hydroxylated) amino acids may be used to constitute sample peptides.

Preferably, all of the sample peptides used are equal in length (i.e. equal in the number of amino acid residue), thus providing for an efficient FNN analysis and extraction of more reliable rules.

The length of each of the sample peptides is not particularly limited. For example, the sample peptides may be 3 to 15 amino acid residues in length. However, taking into consideration that known adhesion peptides are generally composed of 3 to 6 amino acid residues, the sample peptides may preferably be 3 to 8 amino acid residues, more preferably 3 to 6 amino acid residues in length. Thus, use of shorter peptides is preferable since it leads to an increased efficiency of the following FNN analysis and of synthesis of peptides.

An appropriate target for use in the instant affinity assay is selected based on an intended use of the peptide that is designed by the present designing method. The target can be cell, protein, peptide or other biopolymer. In addition, target can be particulate or base (substrate) made of any metal, semiconductor, inorganic material, synthetic polymer, etc. The cell of interest can be any types of cells of mammal (for example, human, monkey, bovine, equine, rabbit, mice, rat, guinea pig, hamster), for example, myocardial cell, smooth muscle cell, adipocyte, fibroblast, bone cell, chondrocyte, osteoclast, parenchyma cell, keratinocyte, epithelial cell (such as dermal epithelial cell, corneal epithelial cell, oral mucous membrane epithelium, follicular epithelial cell, oral mucous membrane epithelial cell, airway mucous membrane epithelial cell, intestinal mucosa epithelial cell), endothelial cell (such as corneal endothelial cell, vascular endothelial cell), nerve cell, glia cell, splenic cell, pancreatic beta cell, mesangial cell, Langerhans cell, liver cell, or precursor cell thereof, or mesenchymal stem cell (MSC), embryonic stem cell (ES cell), embryonic germ cell (EG cell), or adult stem cell. In addition to normal cells, abnormal cells such as cancer cell and HeLa cell, and CHO cell, Vero cell, HEK 293 cell, HepG2 cell, Cos-7 cell, NIH3T3 cell, Sf9 cell and other established strains of cell can be used.

On the other hand, the protein in the present invention can be any receptor protein, ligand protein, antigenic protein, antibody, enzyme, and heat shock protein, for example.

“Affinity assay” refers to a test to detect and evaluate an affinity of a peptide (each of the peptide sequences) toward a given target, and generally involves contacting each peptide with a target followed by washing process which removes any substance that is non-specifically adhered.

Although it is possible to perform an affinity assay for each type of the peptides, the affinity assay can preferably evaluate affinities of a plurality of peptide types toward a given peptide at a time, and thus shorten the operation time. Such an affinity assay can be achieved through use of a peptide chip which has a plurality of peptides immobilized thereon. Peptide chips in general have a plurality of peptides segmented according to each peptide sequence and immobilized on a substrate. Use of such a peptide chip will significantly reduce the operation time. It is preferable to provide a peptide chip with all of the sample peptides in the affinity assay immobilized thereon. However, those sample peptides in question in the affinity assay can be divided into two or more peptide chips. In this case, the peptide chips must be brought into contact with the target under the same condition.

Another advantage of using a peptide chip is that an affinity assay can be performed under a totally identical condition for each of the plurality of peptides for a target, thus yielding highly reliable data.

For example, the instant step can be performed using a peptide chip having a peptide library with random sequences immobilized thereon.

### (2) Selection of Peptide Sequences (FIG. 1 (ii))

Following to step (1), high-affinity and low-affinity peptide sequences are selected (step (2)). In this step, those peptide sequences are selected which exhibited a high affinity and a low affinity based on the affinity data obtained for each of the peptide sequences. The number or ratio of the peptide sequences to be selected are not specifically limited. For example, when the sample peptides used are sorted out in the order of affinity, those sample peptides within the range of the uppermost 1%˜20%, preferably 1%˜10% are selected as high-affinity peptide sequences, and those in the lowermost 1%˜20%, preferably 1%˜10% are selected as low-affinity peptide sequences.

### (3) Transformation to Numerical Data (FIG. 1 (iii))

Following step (2), by digitizing properties of amino acids for each location from N-terminal or C-terminal, each of the selected peptide sequences are transformed into numerical data (step (3)). This step transforms each of the selected peptide sequences into a form that can be analyzed through FNN, and provides data wherein each of the selected sequences has any values representing certain properties with respect to each location thereon.

The certain property can be one or more properties of amino acids, selected from the group consisting of size, hydrophobicity, charges, isoelectric point, presence or absence of branches, presence or absence of sulfur element, presence or absence of hydroxyl group, presence or absence of benzene ring, and presence or absence of heterocycle. Preferably, two or more properties selected from the above group are used. In the instant embodiment, numerical data is provided wherein each of the selected sequences has two or more values with respect to each location of its constituent amino acid.

According to a preferred embodiment of the present invention, three properties of an amino acid, namely size, hydrophobicity and charges are used since these properties are essential and critical in determining the affinity of a peptide as a whole.

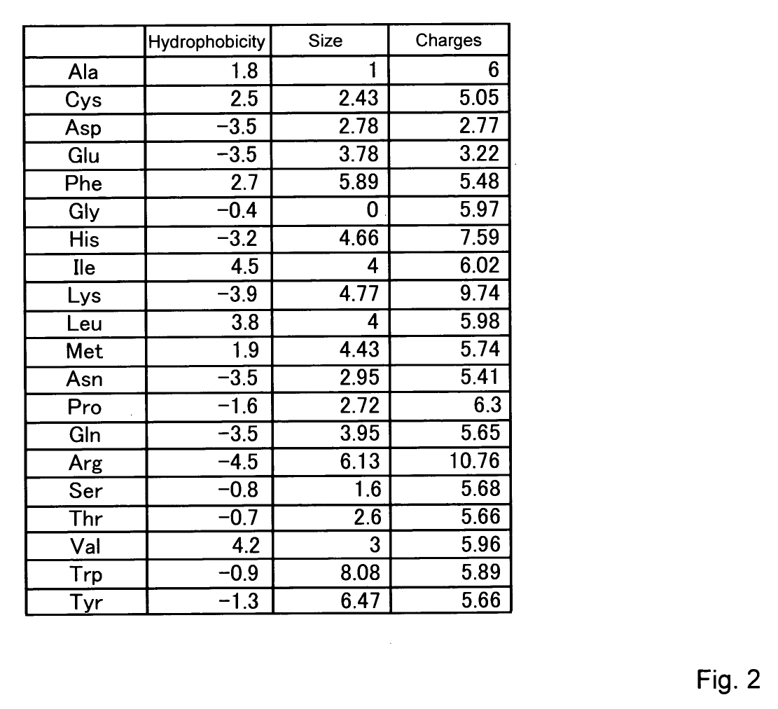

An exemplary digitization of the properties of amino acids is shown in FIG. 2. In FIG. 2, hydrophobicity, size and charges of amino acids are digitized based on known indices (hydrophobicity: Kyte, J. & Doolittle, R. F. (1982) A simple method for displaying the hydropathic character of a protein. J. Mol. Biol. 157, 105-132, size: Fauchere, J. L., Charton, M., Kier, L. B., Verloop, A. & Pliska, V. (1988) Amino acid side chain parameters for correlation studies in biology and pharmacology. Int. J. Peptide Protein Res. 32, 269-278., charges: Zimmerman, J. M., Eliezer, N. & Simha, R. (1968) The characterization of amino acid sequences in proteins by statistical methods. J. Theor. Biol. 21, 170-201))

### (4) Construction of FNN Model (FIG. 1 (iv))

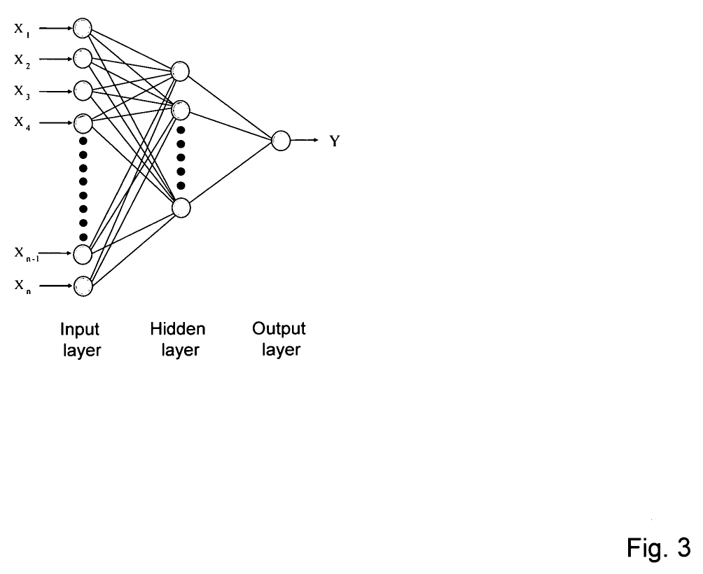

Subsequently, the numerical data obtained in the step of (3) is used as input variables to perform Fuzzy Neural Network (FNN) analysis and construct a prediction model (step (4)). “Fuzzy Neural Network” refers to a combination of “Artificial Neural Network; ANN) and Fuzzy Inference, wherein man-made determination of membership function, which has been a defect of Fuzzy Inference, is avoided by incorporating ANN into the place to effect the determination automatically. ANN (FIG. 3), one of learning machine, is a mathematical model of neural network system in brain in vivo, and has the following characteristics. Learning in the ANN is a process of constructing a model by shifting connection loads interconnecting indicia ∘ in a circuit shown in FIG. 3 by Back propagation or BP method using target output value (teacher values) and learning data (input values; X), such that an actual output value (Y) approximates a teacher value, i.e. an error between the teacher value and the output value (Y) is reduced. Using the BP method, ANN automatically acquires knowledge through learning. Ultimately, data that has not been used in the learning is input to evaluate the universality of the model thus constructed. Determination of membership function, which has been conventionally made by human intuition, is now done by Fuzzy Inference incorporated into the ANN described above, thus allowing for automatic identification of membership function. In such a FNN, BP method, as in ANN, is used to automatically identify and model the input/output correspondence fed to the network by shifting connection loads. FNN analyzes the model after learning and thus provides for acquiring knowledge as a linguistic rule (see FIG. 1 (v)) in Fuzzy Inference readily comprehensible to human. Thus, FNN automatically determines an optimal combination of Fuzzy Inference according to a combination of numerical variables representing properties of amino acids based on their structures and attributes, and, at the same time, estimates affinity of peptides and generates their rules.

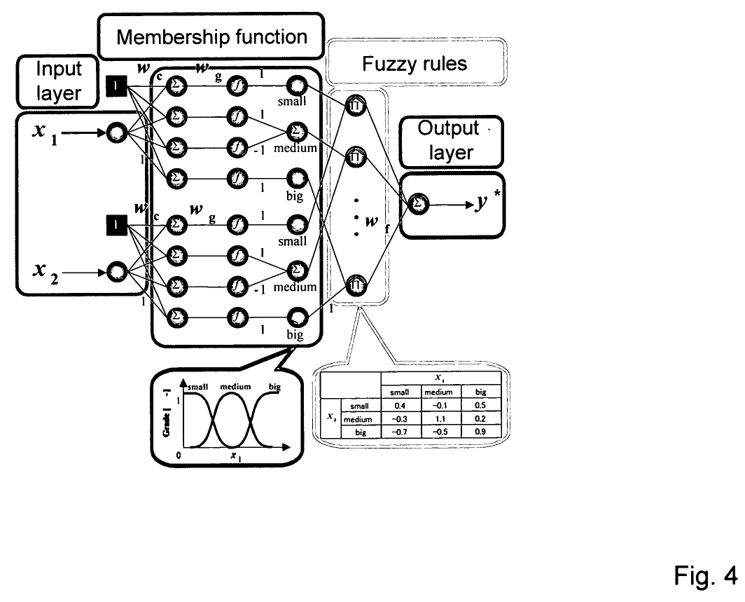

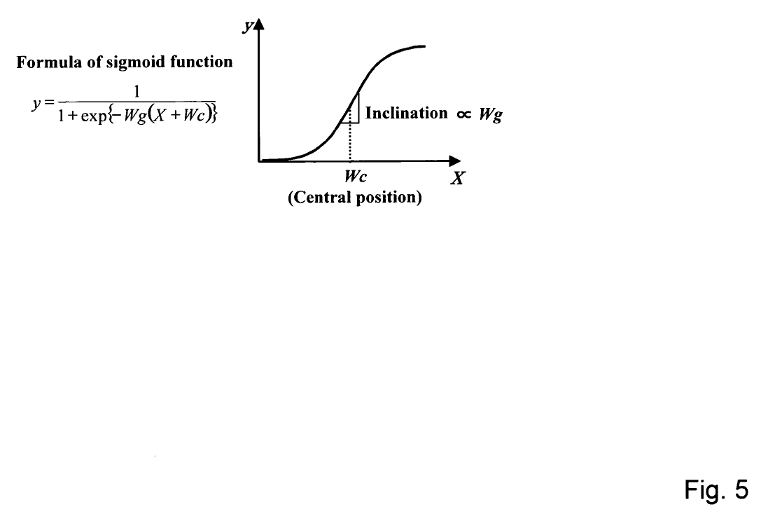

FNN consists of four layers, namely an input layer, membership function part (pre-condition part) for determining parameters Wc and Wg comprised in an sigmoid function, fuzzy rule part (post-condition part) for determining Wf and permitting to extract correspondence between input and output as rules, and output layer (FIG. 4). Connection loads for determining a model structure in FNN consists of Wc, Wg, and Wf. The connection load Wc represents a central location of the sigmoid function used in the membership function, and Wg determines an inclination in the central location (FIG. 5). The connection load Wf represents contribution of each fuzzy region to an estimated result. This Wf can lead to a fuzzy rule.

Wf values, one of the connection loads, are used for generation of fuzzy rules in the FNN analysis. If a Wf value is positive and greater in number, it has a greater contribution to the determination that the unit in quest ion has a high affinity, and a corresponding peptide having any amino acids that meets the rule is determined to have a “high affinity”. In contrast, if a Wf value is negative and smaller in number, it has a greater contribution to the determination that the unit in question has a low affinity, and a corresponding peptide having any amino acids that meets the rule is determined to have a “low affinity”.

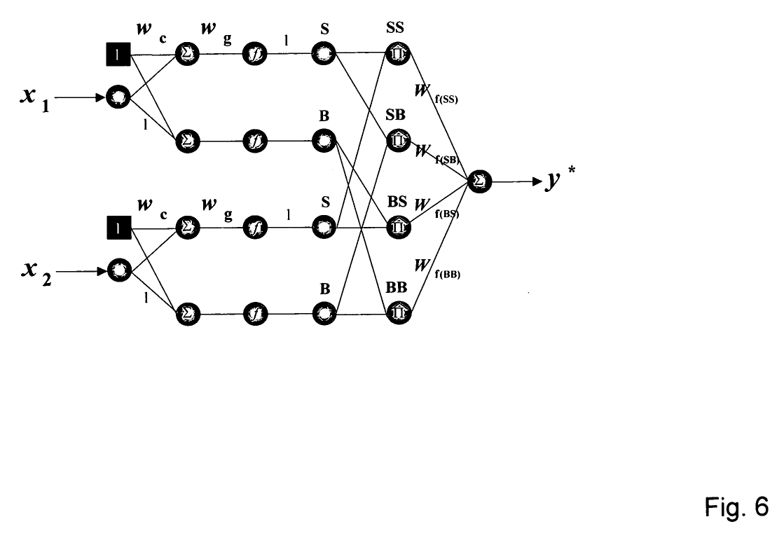

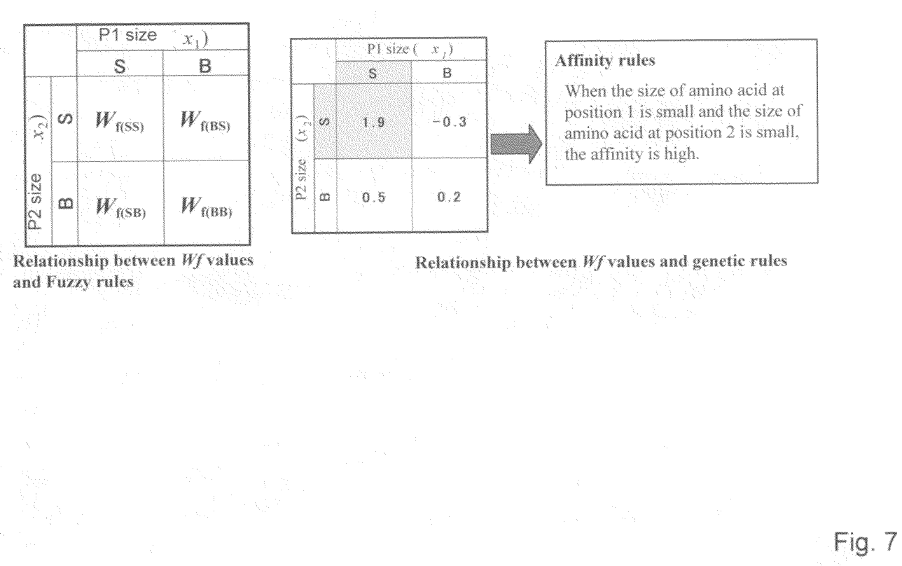

FIG. 6 shows a structure of FNN wherein there are two inputs, and two rules for small (S) and big (B) are provided for each of them. Values entering into SS (when the first input is small, and the second one is small), SB, BS, and BB of the fuzzy rule part are normalized such that a sum of their corresponding values at the four nodes is equal to one (1). Subsequently, the values are multiplied with Wf(SS), Wf(SB), Wf(BS), and Wf(BB), which are then summed up to yield an output value y. An exemplary fuzzy rule for when there are two inputs, and two rules for small (S) and big (B) are provided is shown in FIG. 7.

### (5) Extraction of Rules (FIG. 1 (v))

One or two or more rules wherein amino acids and the properties de scribed above are related to each other on one or more location on sequence according to a prediction model constructed as a result of the above FNN analysis, and thus representing characteristics of high-affinity peptide sequences are extracted (step (5)). Thus, rules representing characteristics of high-affinity peptide sequences are selected using a rule table generated in the FNN analysis. The number of rules extracted is not limited to one. In some instances, a plurality of rules can be extracted. On the other hand, all of the amino acids at any location on the instant peptide sequence are not necessarily related to certain properties. In other words, depending on the rules extracted, the amino acid at least at one location is related to the properties adopted (for example, larger in size, or high in hydrophobicity).

### (6) Designing of Peptides (FIG. 1 (vi))

Once the rule(s) have been extracted, peptides are designed according to those rules (step (6)). If a rule has been extracted which determines that a larger amino acid at the first position (hereinafter referred to as “position 1”) from N-terminal contributes to a high affinity, any one of a plurality of those amino acids which has been classified as “larger in size” in the digitization step of amino acid size is selected as an amino acid to be placed at the position 1. Thus, an amino acid is selected for each and every position according to the rules, thereby obtaining a possible peptide sequence from their combination. Consequently, a peptide sequence can be designed which is expected with a high probability to exhibit a high affinity.

In a preferred embodiment of the present invention, in this step, a plurality of peptides having different peptide sequences are designed according to the extracted rules(s), and the plurality of peptides designed are again subjected to an affinity assay and FNN analysis. In such an embodiment, the steps of (1) to (6) are repeated to optimize the rules, thereby allowing for designing of peptide with a higher affinity and increasing the proportion of high-affinity peptides among all the peptides designed according to the rule(s). The number of repetitions of these steps is not particularly limited. For example, these steps may be repeated 1 to 10 times.

### 2. Method for Preparing High-Affinity Peptides (Production Method)

High-affinity peptides are obtained by actually preparing the peptides designed as described above. Thus, the present invention also provides a method for preparing a high-affinity peptide, comprising preparing the peptide designed according to the designing method described above.

Any known methods of synthesizing peptides (for example, solid-phase synthesis, and liquid-phase synthesis) can be used to prepare the peptide of interest. Any automatic peptide synthesizer may be used to facilitate a ready and fast synthesis of peptide of interest.

Genetic engineering technique may be used to prepare peptides. Specifically, a nucleic acid encoding a peptide according to the present invention may be introduced into an appropriate host cell, and peptides expressed in the transformant recovered to obtain the peptide of interest. The recovered peptide may be purified as appropriate. The recovered peptide may be subjected to any appropriate substitution reaction to convert it into any desired modified peptide.

### 3. Storage Medium Storing a Program for Designing High-Affinity Peptides, and Designing Apparatus

In another aspect, the present invention provides a computer-readable storage medium storing a program for performing a designing method according to the present invention. The storage medium according to the present invention is readable by any universal or dedicated computer, and has a program to perform a method of the present invention stored thereon. The storage medium according to the present invention can be portable or stationary. Exemplary forms of the storage medium are CD-ROM, flexible disk (FD), DVD, hard disk, and semi-conductor memory.

In order to design high-affinity peptides, the program referred to above performs the following steps: (a) digitizing predetermined property(ies) of amino acids for each location from N-terminal or C-terminal to transform into numerical data a plurality of peptide sequences having different affinities toward a given target; (b) performing Fuzzy Neural Network analysis by using the obtained numerical data as input variables to construct a prediction model; and (c) extracting from the constructed prediction model one or two or more rules wherein the amino acids and the properties are related to each other on one or more locations on the sequences, said rules representing the characteristics of high-affinity peptide sequence. The steps of (a), (b) and (c) correspond to the step of (3) (transformation step to numerical data), the step p of (4) (construction step of FNN model) and (5) (extraction step of rules), respectively, of the designing method according to the present invention. The program may be constructed such that a process corresponding to the step of (2) of the designing method of the present invention is performed as well. Specifically, the program in this embodiment performs the step of (i) selecting high-affinity peptide sequences and low-affinity peptide sequences based on affinity data for each of the peptide sequences toward the target, obtained through affinity assay using a plurality of peptides having different sequences and a target, followed by the step of (a′) that corresponds to the step of (a) described above, of digitizing predetermined property(ies) of amino acids for each location from N-terminal or C-terminal to transform into numerical data the high-affinity peptide sequences and low-affinity peptide sequences, and further by the steps of (b) to (d) described above.

In addition, the program may be constructed so as to perform a process corresponding to the step (6) of the designing method of the present invention. Specifically, the program in this aspect performs the steps of (a) to (d) described above, followed by step of (I) designing peptides according to the rules described above.

Moreover, the program may perform both of the steps of (i) and (I). Specifically, one embodiment of the present invention provides a computer-readable storage medium storing a program for performing the steps of (i), (a′), (b) to (d) and (I).

Further, the present invention provides an apparatus for performing the designing method according to the present invention, specifically an apparatus for designing high-affinity peptides. The designing apparatus of the present invention comprises (a) means for digitizing predetermined property(ies) of amino acids for each location from N-terminal or C-terminal to transform into numerical data a plurality of peptide sequences having different affinities toward a given target; (b) means for performing Fuzzy Neural Network analysis by using the obtained numerical data as input variables to construct a prediction model; and (c) means for extracting from the constructed prediction model rules wherein the amino acids and the properties are related to each other on one or more locations on the sequences, said rules representing the characteristics of the high-affinity peptide sequences. The apparatus may perform the step (i) and/or step (I) as well. Namely, the apparatus according to the present invention may comprise means (i) to perform the step (i) and/or means (I) to perform the step (I)

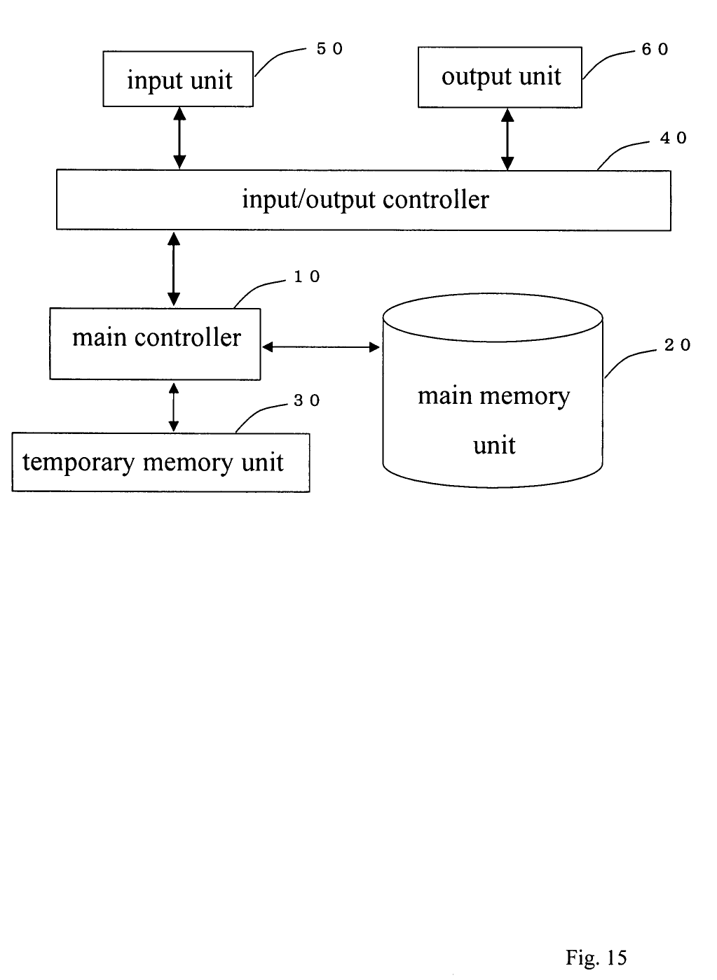

In a preferred embodiment, the designing apparatus of the present invention is a computer storing the program according to the present invention in the memory unit such as hard disk. An exemplary hardware structure of the designing apparatus of the present invention is shown in FIG. 15. In such a structure, the apparatus comprises a main controller 10 for controlling the entire apparatus, a main memory unit 20 and a temporary memory unit 30 both connected to the main controller 10, and an input unit 50 and an output unit 60 both connected via input/output controller 40 to the main controller 10.

The main controller 10 comprises an internal memory for storing OS (Operating System) and other controlling programs, programs defining various processing procedures, other necessary data.

The main memory unit 20 stores a program for performing the process according to the present invention. Specifically, a program (1) for digitizing predetermined property(ies) of amino acids for each location from N-terminal or C-terminal to transform into numerical data a plurality of peptide sequences having different affinities toward a given target; (2) for performing Fuzzy Neural Network analysis by using the obtained numerical data as input variables to construct a prediction model; and (3) for extracting from the constructed prediction model one or two or more rules wherein the amino acids and the properties are related to each other on one or more locations on the sequences, said rules representing the characteristics of the high-affinity peptide sequences. The processes (1) to (3) may be performed by two or more programs (For example, a program for performing the process of (1), a program for performing the processes of (2) and (3), and a program for controlling these programs may be used). The main memory unit 20 stores, in addition the programs described above, various database, tables, and files (for example, data file wherein the amino acids and properties (such as size, hydrophobicity, charges, isoelectric point, presence or absence of branch) are related to each other. See FIG. 2. The data file will be hereinafter referred to as “amino acid index file”).

The temporary memory unit 30 temporarily stores, for example, input data and calculated numerical data.

The input unit 50 comprises, for example, a pointing device such as a keyboard and mouse. The output unit 60 comprises, for example, a display and printer.

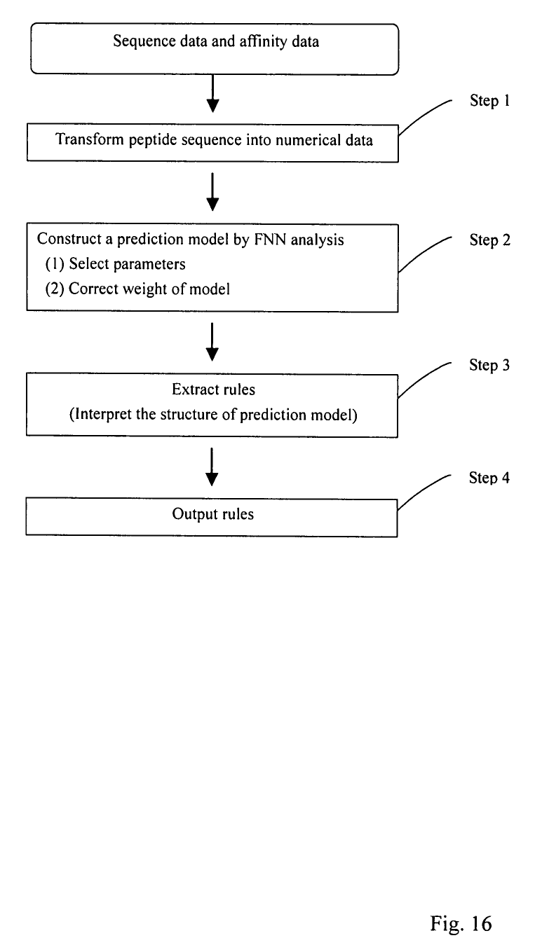

Referring to FIG. 16, the designing apparatus in the form of hardware structure described above receives via the input unit 50 such as a keyboard high-affinity peptide sequence data (sequence information) and low-affinity peptide sequence data (sequence information), and affinity data (teacher signal) for each sequence which have been obtained by an affinity assay. In response, the main controller 10 performs data processing (such as sorting, editing of redundant information, and clustering) according to a predetermined program instruction stored in the main memory unit 20 to digitize predetermined property(ies) of amino acids for each location from N-terminal or C-terminal to transform into numerical data a plurality of peptide sequences having different affinities toward a given target (step 1). Amino acid index files stored in the main memory unit 20 are referenced for this processing. The numerical data obtained is stored in the temporary memory unit 30. Alternatively, data such as sequence information may be read from, for example, portable storage medium.

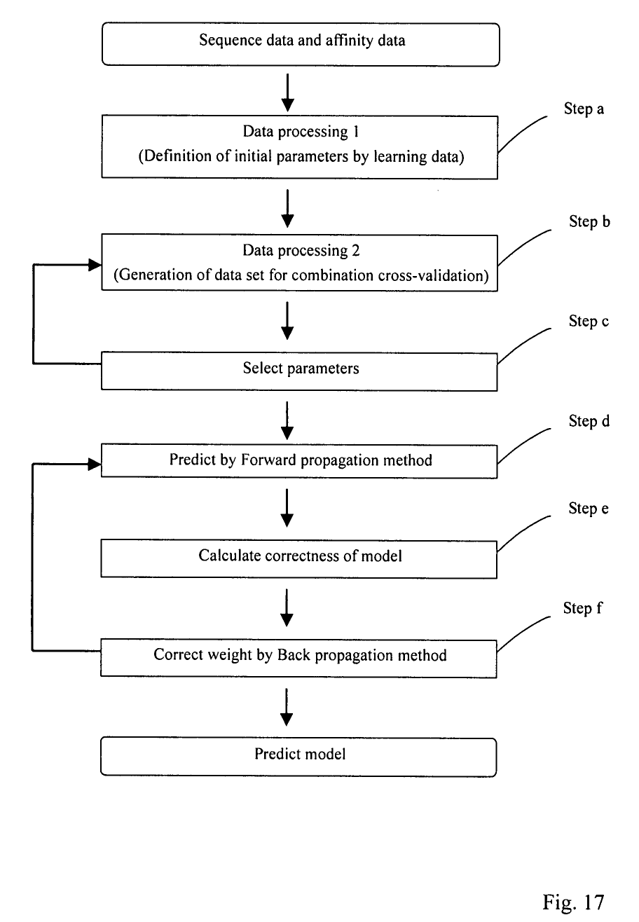

The main controller 10 then accesses the numerical data stored in temporary memory unit 30 according to the program stored in the main memory unit 20, and performs the Fuzzy Neural Network analysis (FNN analysis) b y using the numerical data as input variables to construct a prediction model (step 2). This step generally consists of two processes: (1) selection of parameters, and (2) correction of weight of models. An exemplary processing procedure (algorithm) by software for constructing prediction models through the FNN analysis is shown in FIG. 17. First, sequence information data and affinity data (teacher data) are processed, and initial parameters are defined by learning data (step a). Subsequently, data processing at the second phase is performed to generate data set for combination cross validation (step b) and to select parameters (step c). The steps b and c are repeated until an optimal condition is established. Then, the selected parameters are used to repeat the prediction by Forward propagation method (step d), calculation of correctness (such as minimal errors and correctness) of models (step e), and correction of weight by Pack propagation method (step f), until an optimal condition is established and a prediction model is finally constructed.

The data of the prediction model thus constructed is stored in the temporary memory unit 30. The main controller 10 then accesses the data of the prediction model stored in the temporary memory unit 30, according to an instruction of the program stored in the main memory unit 20, and extracts, based on the prediction model, one or two or more rules representing the characteristics of high-affinity peptide sequences (rules wherein the amino acid and the properties are related to each other on one or more locations on the sequences (step 3 in FIG. 16). The rules extracted are stored in the main memory unit 20 and the temporary memory unit 30, and outputs to an out put unit 60 automatically or in response to a call from an operator (step 4). The main memory unit 20 or the temporary memory unit 30 accumulate, in addition to the data files relating to the rules above, data files representing prediction models, and files relating to correctness of the prediction models, as necessary. These files are output to the output unit 60 automatically or in response to a call from an operator.

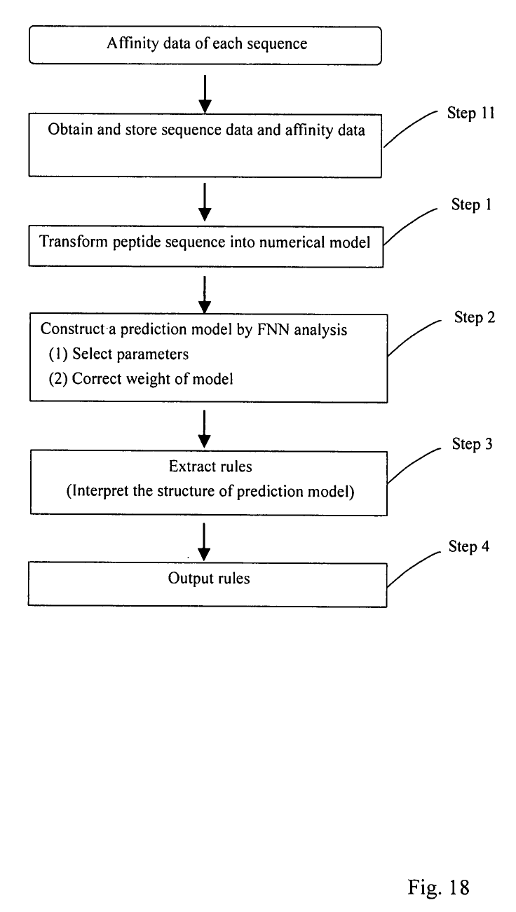

An embodiment of the present designing apparatus will be now described in terms of its hardware structure and processing which incorporates into the hardware the means (i) in addition to the means (a) to (c). The hardware structure according to this embodiment of the designing apparatus is similar to that of the designing apparatus described above, except that the main memory unit 20 stores, in addition to the program for performing steps a to f, a program for determining the degree of affinity, before the step of a. A shown in FIG. 18, in the instant designing apparatus, in response to an input of affinity data for each sequence toward a given target that has been obtained by affinity assay, the main controller 10 selects high-affinity peptide sequences and low-affinity peptide sequences according to the instruction from the program described above stored in the main memory unit 20. The result (specifically, high-affinity peptide sequence data (sequence information), low-affinity peptide sequence data (sequence information), and affinity data for each sequence) is stored in the temporary memory unit 30 (step 11). Subsequently, these data is utilized to perform steps 1 to 4.

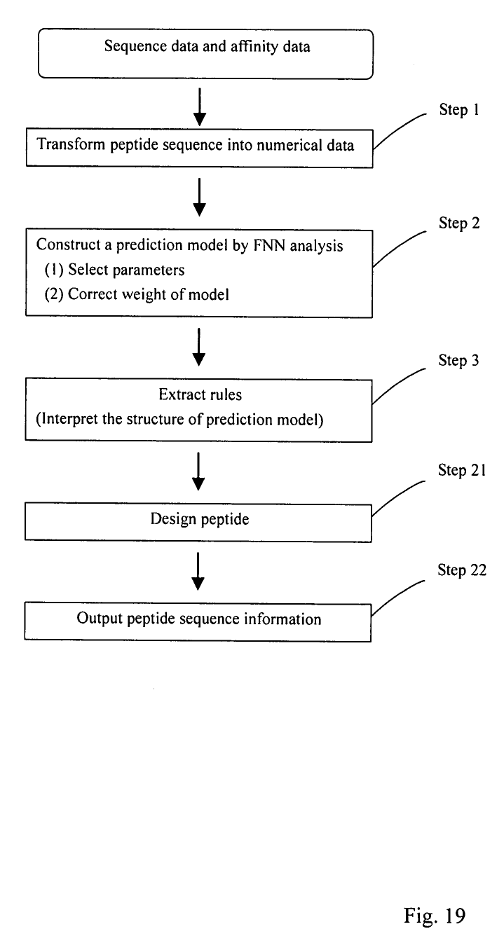

An additional embodiment of the present designing apparatus will be now described in terms of its hardware structure and processing which incorporates into the hardware the means (I) in addition to the means (a) to (c). The hardware structure according to this embodiment of the designing apparatus is similar to that of the designing apparatus described above, except that the main memory unit 20 stores, in addition to the program for performing steps a to f, a program for designing peptides according to the extracted rules, after the step of f. As shown in FIG. 19, in the instant designing apparatus, after performing the steps 1 to 3 as in the designing apparatus described above, the main controller 10 receives the instruction from the above program, and designs peptides according to the rules stored in the temporary memory unit 30 (step 21). The data (sequence information) of the designed peptides is stored in the main memory unit 20 or temporary memory unit 30, and, are output to the output unit 60 automatically or in response to a call from an operator (step 22).

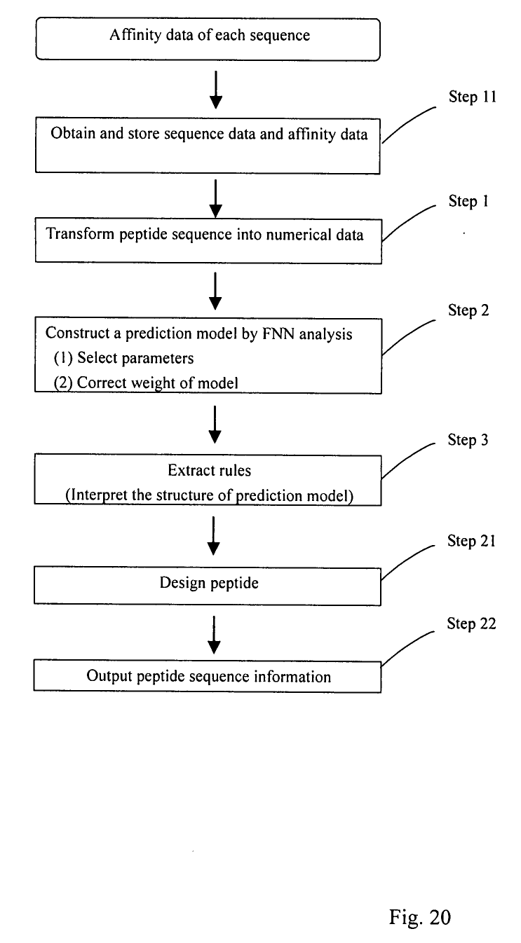

An additional embodiment of the present designing apparatus will be now described in terms of its hardware structure and processing which incorporates into the hardware the means (i) and (I) in addition to the means (a) to (c). The hardware structure according to this embodiment of the designing apparatus is similar to that of the designing apparatus described above, except that the main memory unit 20 stores, in addition to the program for performing steps a to f, a program for determining a degree of affinity before the step a, and performing the step of designing peptides according to the extracted rules, after the step of f. As shown in FIG. 20, the instant designing apparatus performs the processing in the order of step 11, steps 1 to 3, steps 21 and 22.

### 4. High-Affinity Peptides

The present inventors, as shown later Examples, have succeeded in actually designing peptides that exhibit a high affinity through the designing method according to the present invention. Based on this achievement, the present invention in another aspect provides high-affinity peptides comprising an amino acid sequence shown in any one of SEQ ID NOs:1-70. Sequences of these peptides are shown hereinafter. Peptides according to SEQ ID Nos.: 1-42 correspond to those which showed 1.5 or higher relative fluorescence intensity in the affinity assay among the peptides synthesized according to the rules (IV) extracted in the embodiment which will be described later. Similarly, peptides according to SEQ ID Nos.:43-70 correspond to those which showed 1.5 or higher relative fluorescence intensity in the affinity assay among the peptides synthesized according to the rules (V) extracted in the embodiment which will be described later.

| | |
|---|---|
| GKFQ, | (SEQ ID NO.:1) |
| MKHT, | (SEQ ID NO.:1) |
| PKYL, | (SEQ ID NO.:3) |
| MKKN, | (SEQ ID NO.:4) |
| ARYD, | (SEQ ID NO.:5) |
| VKRG, | (SEQ ID NO.:6) |
| PRFQ, | (SEQ ID NO.:7) |
| IRHM, | (SEQ ID NO.:8) |
| MRKV, | (SEQ ID NO.:9) |
| PKHI, | (SEQ ID NO.:10) |
| AKKG, | (SEQ ID NO.:11) |
| VKWS, | (SEQ ID NO.:12) |
| AKRT, | (SEQ ID NO.:13) |
| ARHG, | (SEQ ID NO.:14) |
| MRHC, | (SEQ ID NO.:15) |
| FRHI, | (SEQ ID NO.:16) |
| PKKV, | (SEQ ID NO.:17) |
| VKMS, | (SEQ ID NO.:18) |
| AKHA, | (SEQ ID NO.:19) |
| FKMV, | (SEQ ID NO.:20) |
| IRKE, | (SEQ ID NO.:21) |
| AKWQ, | (SEQ ID NO.:22) |
| VKHI, | (SEQ ID NO.:23) |
| MKWV, | (SEQ ID NO.:24) |
| MKFN, | (SEQ ID NO.:25) |
| ARYI, | (SEQ ID NO.:26) |
| MKMV, | (SEQ ID NO.:27) |
| PRRT, | (SEQ ID NO.:28) |
| IRRG, | (SEQ ID NO.:29) |
| IKRT, | (SEQ ID NO.:30) |
| AKME, | (SEQ ID NO.:31) |
| MRMV, | (SEQ ID NO.:32) |
| MRKL, | (SEQ ID NO.:33) |
| FKWP, | (SEQ ID NO.:34) |
| ARYA, | (SEQ ID NO.:35) |
| MKRP, | (SEQ ID NO.:36) |
| IRHP, | (SEQ ID NO.:37) |
| MKYQ, | (SEQ ID NO.:38) |
| VKHP, | (SEQ ID NO.:39) |
| VRMT, | (SEQ ID NO.:40) |
| FKRL, | (SEQ ID NO.:41) |
| FKWN, | (SEQ ID NO.:42) |
| KGMH, | (SEQ ID NO.:43) |
| KGIR, | (SEQ ID NO.:44) |
| RSVF, | (SEQ ID NO.:45) |
| RGVH, | (SEQ ID N0.:46) |
| KGTR, | (SEQ ID NO.:47) |
| HHYH, | (SEQ ID NO.:48) |
| RIFF, | (SEQ ID NO.:49) |
| KQCR, | (SEQ ID NO.:50) |
| RPVH, | (SEQ ID NO.:51) |
| RFYR, | (SEQ ID NO.:52) |
| KSSY, | (SEQ ID NO.:53) |
| RECY, | (SEQ ID NO.:54) |
| RYNH, | (SEQ ID NO.:55) |
| HLVK, | (SEQ ID NO.:56) |
| RTWR, | (SEQ ID NO.:57) |
| HAQK, | (SEQ ID NO.:58) |
| HFFR, | (SEQ ID NO.:59) |
| KWGK, | (SEQ ID NO.:60) |
| KPDK, | (SEQ ID NO.:61) |
| HMMR, | (SEQ ID NO.:62) |
| HCMY, | (SEQ ID NO.:63) |
| RAMR, | (SEQ ID NO.:64) |
| RVWK, | (SEQ ID NO.:65) |
| KENK, | (SEQ ID NO.:66) |
| REWR, | (SEQ ID NO.:67) |
| HQWY, | (SEQ ID NO.:68) |
| RHDF, | (SEQ ID NO.:69) |
| KACW, | (SEQ ID NO.:70) |

These peptides are promising for use as an adhesion peptide in, for example, culture base, transplant material (as a substrate for cell transplant and as carrier in DDS, for example), and cell targeting. Especially, peptides shown in any one of SEQ ID NO.:1-8 and 43-48 (those which showed 2.0 or higher relative fluorescence intensity in the affinity assay) have an extremely high affinity, and are greatly expected to provide applicability as adhesion peptide.

The peptides described above, as long as it maintains its high adhesiveness, may be modified in any manner. Specifically, an aspect of the present invention provides a modified form of the peptides described above (hereinafter referred to as “modified peptide”). The “modified peptide” according to the present invention is a compound which at least in part differs from the peptide of interest in its structure by having a portion (or portions) of its basic peptide structure replaced with any other group, added with any other molecule, or modified in any other manner. Those who skilled in the art will be able to design a replaced or any other modified form of the basic peptides described above through use of known or routine means. Further, any modified form of the peptides described above may be prepared through use of known or routine means based on such a design.

An exemplary modified peptide would be a peptide derivative wherein a portion (an atom or group) of any side chains on an amino acid residue constituting a peptide is replaced with any other atom or group. Such a peptide derivative can be prepared through any manufacturing process designed so as to obtain the peptide derivative of interest as a final product. Accordingly, if the peptide derivative of interest is a peptide having a portion (for example a group which is a part of a side chain) thereof apparently replaced with a certain group, that peptide derivative of interest may be manufactured through a displacement reaction using a particular group and an apparently basic peptide as a starting material, or through any appropriate displacement reaction or any other process (or optionally processes) using any peptide having any other structure as a starting material.

The atom or groups for displacement may be, for example, hydroxyl, halogen (fluorine, chlorine, buromine, iodine, for example), alkyl (methyl, ethyl, n-propyl, isopropyl, for example), hydroxyalkyl (hydroxymethyl, hydroxyethyl, for example), alkoxy (methoxy, ethoxy, for example), acyl (formyl, acetyl, maronyl, benzoyl, for example).

Modified peptides further include those which have a functional group within its constituting amino acid residue protected by any appropriate protective group. Protective groups for use for this purpose may be, for example, acyl, alkyl, monosaccharide, oligosaccharide, and polysaccharide. Such protective groups are linked via, for example, amide linkage, ester linkage, urethane linkage, and urea linkage depending on the site on the peptide to attach the protective group as well as the type of protective group used.

Other examples of modified peptides are those which are modified through addition of sugar chain. Further, those various peptide derivatives which are substituted on their N-terminal or C-terminal with, for example, any other atom and classified into alkylamine, alkylamide, sulfynyl, sulfonylamide, halide, amide, aminoalcohol, ester, aminoaldehyde, etc. are considered as modified peptides.

Still further examples of modified peptides are labeled peptides. For example, those peptides which are labeled on their N-terminal with biotin or FITC, or with any fluorescent substance correspond to labeled peptides.

Any combination among the modifications described above may be possible to yield peptide derivatives as modified peptides according to the present invention.

### EXAMPLES

### 1. Design of High-Affinity Peptides Targeting Mouse Fibroblast

A strategy for designing peptides that exhibit a high affinity toward a given target is shown in FIG. 1. The instant strategy consists of six steps. First, a peptide library of random sequence is generated on a peptide array, and an affinity assay is performed using cells to obtain affinity data for each peptide sequence ((i), (ii)). High affinity peptide sequences and low-affinity peptide sequences are selected from the data obtained (ii). The amount of characteristics (size, hydrophobicity and charges) of the amino acid at each position on the selected peptides are digitized, and the peptide sequences are transformed into numerical data (iii). The numerical data is then input into the FNN and subjected to analysis (iv). Based on the prediction model constructed by the FNN, any characteristics found in terms of sequence found in the high-affinity peptide sequences are extracted as rules (v). Peptides are de signed according to these rules (vi). The peptide thus designed are synthesized on arrays, and an affinity assay is performed. The steps (i) to (vi) are repeated to refine (optimize) the rules.

In order to validate the strategy above, peptides were experimentally designed which have an affinity toward mouse fibroblast as a model. In the experiment, rules were extracted also for low-affinity peptide sequences in step (v), and the peptides that had been designed according to those rules served as a control for comparison.

### (1) Materials and Methods

### (1-1) Cell and Culture Medium

Mouse fibroblasts NIH/3T3(American Type Culture Collection, Manassas, Va., USA) were cultured in DMEM (Dulbeccos' Minimum Eagle's Medium) (Gibco, Gaithersgurg, Md.) supplemented with 10% Fetal bovine serum (SIGMA), 100 μg/ml penicilin/streptomycin (Gibco, Gaithersgurg, Md.), and non-essential amino acids (Gibco, Gaithersgurg, Md.) 10 ml, at 37° C., under 50% CO₂.

### (1-2) Production of Peptide Chips

Peptide chips of random sequences of four residue—were produced by known Fmoc solid-phase synthesis, subjected to a short sterilization by methanol, and used for cell assay.

### (1-3) Induction of Cell Adhesion Using Peptide Chips (Affinity Assay)

Peptide chips were seeded with cells, and cultured for a short time at 37° C. under 5% CO₂, followed by washes with saline to remove non-adhered cells. Adhered cells were evaluated using fluorescent dye calcein AM (Molecular Probes, Leiden, Netherland) (Ex: 485 nm, Em: 538 nm) as Relative cell adhesion with the fluorescence intensity on cellulose membrane having no peptide synthesis taking place at 1.0.

### (1-4) Construction of Adherent Peptide Prediction Model Using FNN

In order to find any sequence characteristics to induce cell adhesion by peptide and to design any novel peptides, data mining processing is needed which exploit the characteristics from extensive data. In the present strategy, the FNN has been focused on which allows for simultaneous performance of prediction of cell adhesion and extraction of sequence characteristics (rules). Thus, the FNN was performed using adherent peptides (positive data) obtained as a result of affinity assay (cell assay) using peptide chips as well as non-adherent peptides (negative data). As indicators of the properties of peptides, size, hydrorophobicity and charges of an amino acid at each position were utilized (FIG. 2). Indices by Zimmerman (Zimmerman, J. M., Eleizer, N., and Simha, R.: The characterization of amino acid sequences in proteins by statistical methods. J. Theor. Biol., 21, 170-201(1968)), by Kyte (Kyte, J., and Doolittle, R. F.: A simple method for displaying the hydropathic character of a protein. J. Mol. Biol., 157, 105-132(1982)), and by Fauchere (Fauchere, J. L., Charton, M., Kier, L. B., Verloop, A., and Pliska, V.: Amino acid side chain parameters for correlation studies in biology and pharmacology. Int. J. Peptide Protein Res., 32, 269-278(1988)) were used for size, hydrophobicity and charges, respectively. The positions of amino acids are designated as P1 (the first position from N-terminal), P2 (the second position from N-terminal), P3 (the third position from N-terminal) and P4 (the fourth position from N-terminal) sequentially from N-terminal.

A subject of to what degree input variables representing properties of an amino acid on each position “contribute to cell adhesion” was brought to result in a subject of to what degree of accuracy the FNN constructed using the input variables can identify a positive or negative data, and the accuracy was used as evaluation value. In the present study, an input value for positive data was +1, and that for negative data was −1. When an output value is >0 for positive data and <0 for negative data, the output value was determined to be correct, and the ratio of the number of correct outputs was assumed as the accuracy.

When the analysis was performed using all of the data, the data were divided into modeling data for use in selecting variables, and blind data for use in evaluating the universality of the variables. In performing four-divided cross validation, all of the data was divided into four groups such that the numbers of positive data and negative data are uniform among each data set, and three of them were used as modeling data, and the remaining one as blind data.

In selecting input variables, variable incrementation was used, modeling data for use in the variable selection was divided into four groups, the three of which were used as learning data, and the remaining one was used as evaluation data. The learning data is provided for optimizing parameters in the FNN for certain input variables. The evaluation data is provided for a voiding construction of a model specialized for the learning data (over-learning), and is used for evaluating an FNN model for each and every learning that has occurred.

In order to avoid over-learning, data divided among the modeling data are used once for evaluation without exception (cross-validation). For all of the variables, reference values obtained using each of the variables are calculated for each of the learning data and evaluation data, and those variables which yielded a maximal average value I of the reference values were selected as optimal variables. The second and further input variables, when they are selected, are selected in the similar manner to the above, with the first variable fixed.

Three variables were selected according to the operation described above from among the modeling data, and a model constructed was evaluated using the blind data which had not been used in the modeling. In addition, for avoiding any bias in selection of variables among data set, all of the data were divided into modeling data and blind data multiple times, variables selected for each of them, and those variables which had been selected most frequently were used as input variables for use in constructing a model. The model constructed was represented as a fuzzy rule, and a sequence rule table for predicting positive peptides and negative peptides was plotted based on Wf values and if-then rule table.

### (2) Results

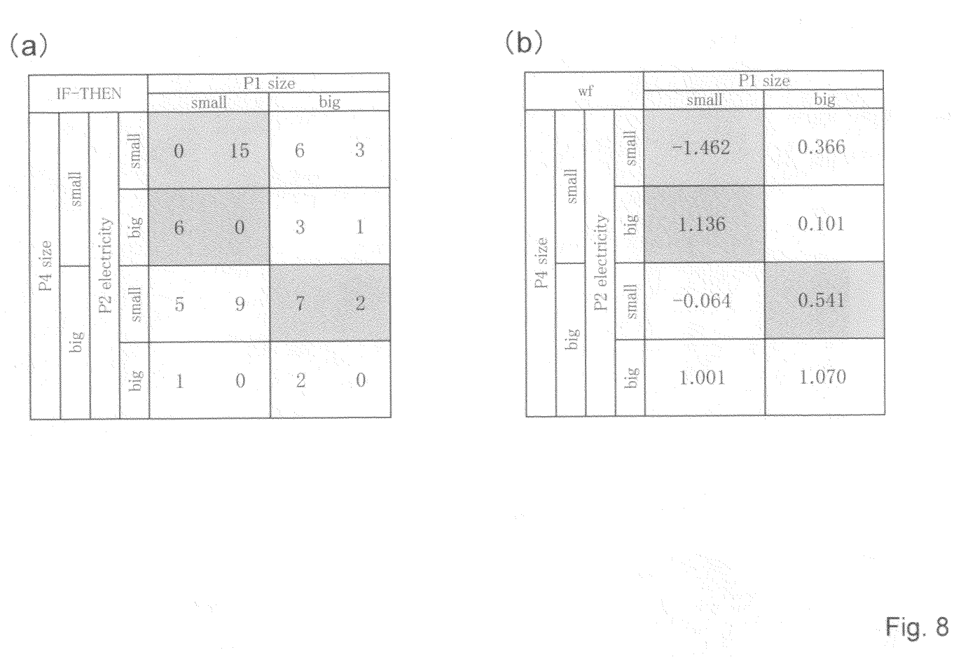

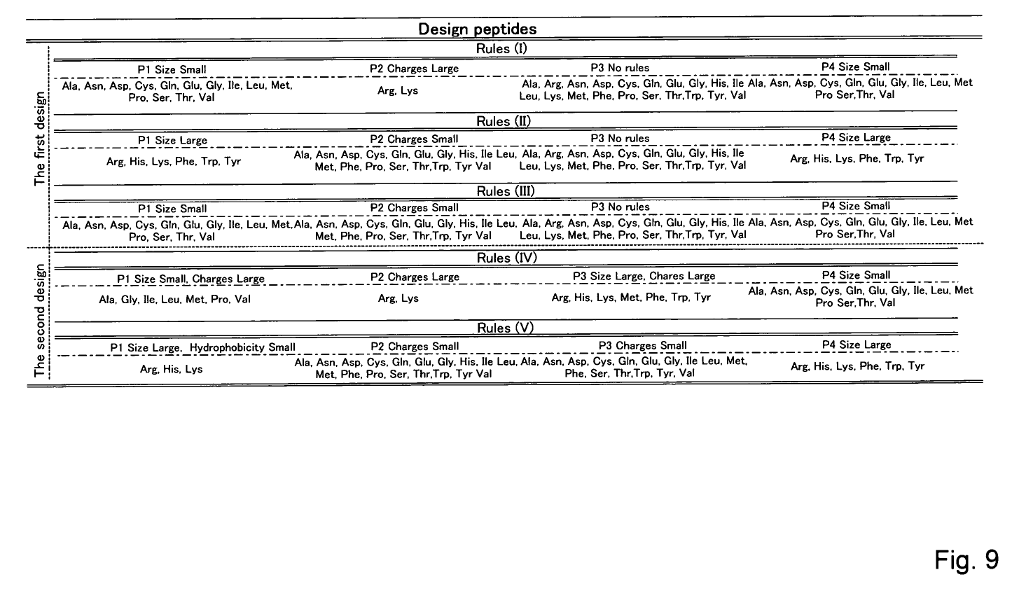

Sequence rule tables generated by the first FNN analysis are shown in FIG. 8. (a) is a if-then rule table, and (b) is a table showing Wf values. As shown, rule tables are generated based on the size of amino acid at P1, charges of amino acid at P2 and the size of amino acid at P4. From the rule tables, two rules (positive rules) showing the characteristics of a high affinity (rule (I) and rule (II) of FIG. 9) and one rule (negative rule) showing the characteristics of a low affinity (rule (III) of FIG. 9) are extracted. A set of values within each cell in the if-then rule table are the numbers of adhesion peptides (positive data; left) and non-adhesion peptides (negative data; right) which fulfill the characteristics related to that cell. In the FNN analysis, since thresholds are set for each location of an amino acid and for each parameter, selected amino acids may differ even if under apparently identical conditions. For example, the conditions relating to the size at P1 and the size at P2 are identical (small), different amino acids are selected.

In order to refine (optimize) the rules, the peptides synthesized according to the positive rules described above which had been extracted from the result of the first FNN analysis were used to repeat the cycle of affinity as say and FNN analysis. 270 types of peptide and 270 types of peptide according to the rule (I) and (II), respectively, were synthesized and subjected to affinity assay.

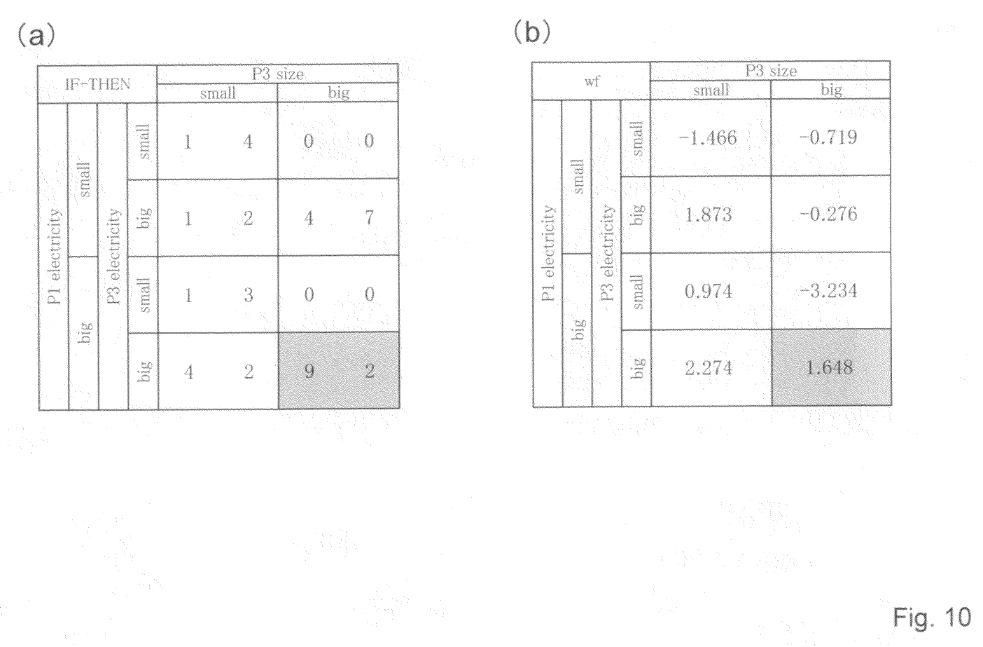

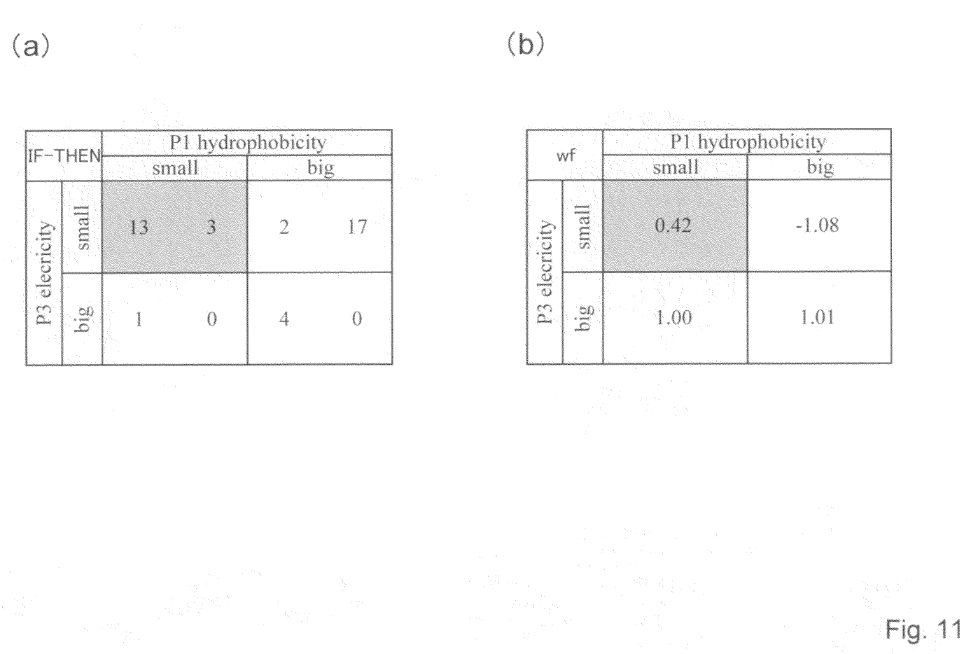

Sequence rule tables generated by the second FNN analysis are shown in FIG. 10 (for peptides according to the rules (I)) and FIG. 11 (for peptides according to the rules (II)). The following rules (IV) (See FIG. 9) are extracted as positive rules from the sequence rule table shown in FIG. 10, wherein P2 and P4 are not applied with any new rules, but with a rule that corresponds to the rule (I).

The following rules (V) (See FIG. 9) are extracted as positive rules from the sequence rule table shown in FIG. 11, wherein P2 and P4 are not applied with any new rules, but with a rule that corresponds to the rule (I).

### <Rules (IV)>

At P1, with small size and large charges: Ala, Gly, Ile, Leu, Met, Pro, or Val

At P2, with large charges: Arg or Lys

At P3, with large size and large charges: Arg, H is, Lys, Met, Phe, Trp, or Tyr

At P4, with small size: Ala, Asn, Asp, Cys, Gin, Glu, Gly, Ile, Leu, Met, Pro, Ser, Thr, or Val

### <Rules (V)>

At P1, small size and low hydrophobicity: Arg, His, [symbol] Lys

At P2, with small charges: Ala, Asn, Asp, Cys, Gln, Glu, Gly, His, Ile, Leu, Met, Phe, Pro, Ser, Thr, Trp, Tyr, or Val

At P3, with small charges: Ala, Asn, Asp, Cys, Gln, Glu, Gly, Ile, Leu, Met, Phe, Ser, Thr, Trp, Tyr, or Val

At P4, with large size: Arg, His, Lys, Phe, Trp, or Tyr 50 types of peptides comprising peptides sequences according to the rule (IV) and 50 types of peptides comprising peptides sequences according to the rule (V) were synthesized and their cell adhesiveness was evaluated. The evaluation method of cell adhesiveness was performed according to the methods described above (1-3).

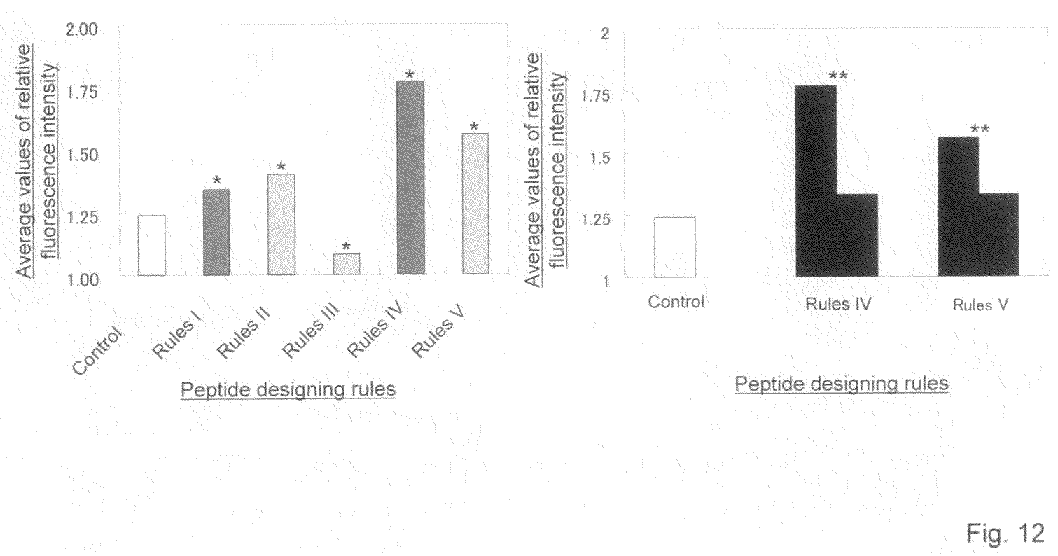

Results of the cell adhesiveness assay is shown in FIG. 12, wherein graph (a) compares the test results among the rules (I) to (V), and graph (b) shows the assay results under the rule (IV) and (V) (shaded), and under the opposite rules (slashed). Control tests were performed using peptides which meet with none of the rules (random sequence peptides). An ordinate in each graph shows an average value of relative fluorescence intensity.

It can be understood from FIG. 12 that peptides showing a high affinity (adherent peptides) under a serum-free condition can be designed according to the positive rules (I), (II), (IV), and (V). Specifically, under the rules (IV), and (V), average values of relative fluorescence intensity (rules (IV): 1. 77, rules (V): 1.56) are obviously increased over that of the control (1.24). In addition, since the corresponding values for the rules (IV) and (V) are significantly higher than those for the rules (I) and (II), it can be concluded that repetition of the cycles of affinity assay and FNN analysis results in refining of the rules (optimization), thereby yielding peptides with a higher affinity.

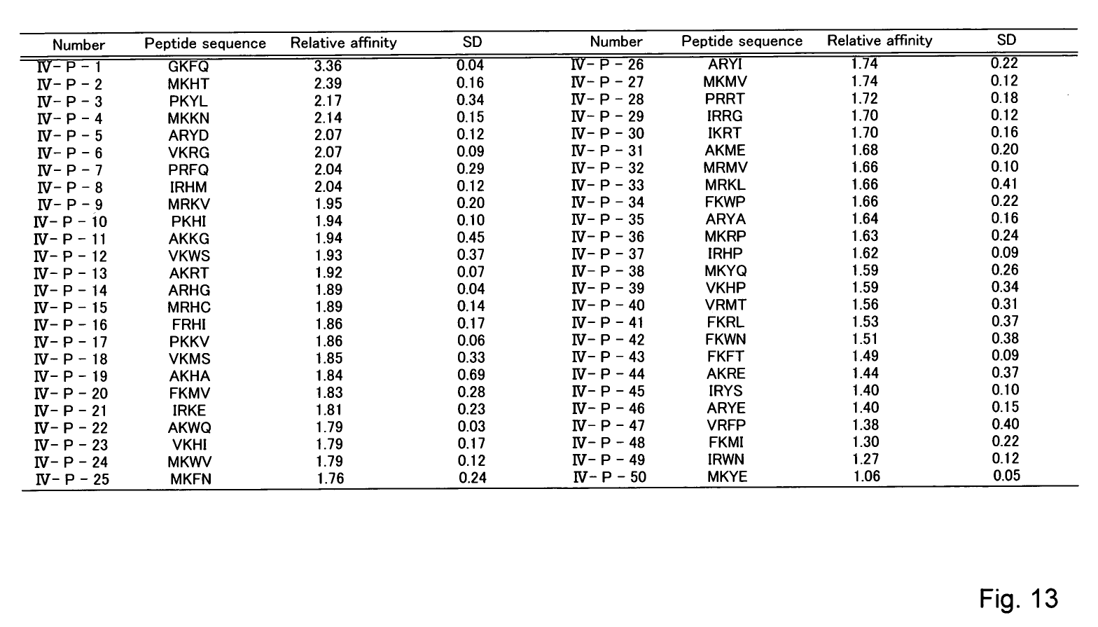

FIG. 13 shows the peptides synthesized according to the rules (IV) and arranged in the degree of affinity, from the one with a greatest affinity at the top. Approximately 80% of the peptides synthesized according to the rules (IV) showed 1.5 or higher relative fluorescence intensity values.

| | |
|---|---|
| GKFQ, | (SEQ ID NO.:1) |
| MKHT, | (SEQ ID NO.:1) |
| PKYL, | (SEQ ID NO.:3) |
| MKKN, | (SEQ ID NO.:4) |
| ARYD, | (SEQ ID NO.:5) |
| VKRG, | (SEQ ID NO.:6) |
| PRFQ, | (SEQ ID NO.:7) |
| IRHM, | (SEQ ID NO.:8) |
| MRKV, | (SEQ ID NO.:9) |
| PKHI, | (SEQ ID NO.:10) |
| AKKG, | (SEQ ID NO.:11) |
| VKWS, | (SEQ ID NO.:12) |
| AKRT, | (SEQ ID NO.:13) |
| ARHG, | (SEQ ID NO.:14) |
| MRHC, | (SEQ ID NO.:15) |
| FRHI, | (SEQ ID NO.:16) |
| PKKV, | (SEQ ID NO.:17) |
| VKMS, | (SEQ ID NO.:18) |
| AKHA, | (SEQ ID NO.:19) |
| FKMV, | (SEQ ID NO.:20) |
| IRKE, | (SEQ ID NO.:21) |
| AKWQ, | (SEQ ID NO.:22) |
| VKHI, | (SEQ ID NO.:23) |
| MKWV, | (SEQ ID NO.:24) |
| MKFN, | (SEQ ID NO.:25) |
| ARYI, | (SEQ ID NO.:26) |
| MKMV, | (SEQ ID NO.:27) |
| PRRT, | (SEQ ID NO.:28) |
| IRRG, | (SEQ ID NO.:29) |
| IKRT, | (SEQ ID NO.:30) |
| AKME, | (SEQ ID NO.:31) |
| MRMV, | (SEQ ID NO.:32) |
| MRKL, | (SEQ ID NO.:33) |
| FKWP, | (SEQ ID NO.:34) |
| ARYA, | (SEQ ID NO.:35) |
| MKRP, | (SEQ ID NO.:36) |
| IRHP, | (SEQ ID NO.:37) |
| MKYQ, | (SEQ ID NO.:38) |
| VKHP, | (SEQ ID NO.:39) |
| VRMT, | (SEQ ID NO.:40) |
| FKRL, | (SEQ ID NO.:41) |
| FKWN, | (SEQ ID NO.:42) |

Among these, GKFQ(SEQ ID NO.:1), MKHT(SEQ ID NO.:1), PKYL(SEQ ID NO.:3), MKKN(SEQ ID NO.:4), ARYD(SEQ ID NO.:5), VKRG(SEQ ID NO.:6), PRFQ(SEQ ID NO.:7), IRHM(SEQ ID NO.:8) have 2.0 or higher relative fluorescence intensity values, thus revealing an extremely high affinity.

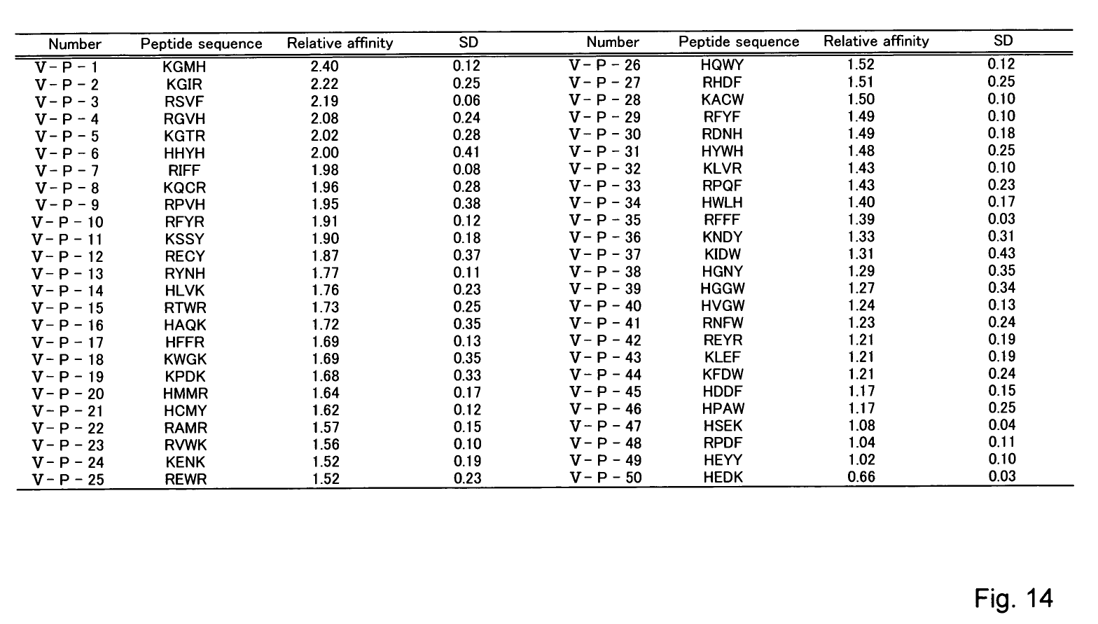

On the other hand, as shown in FIG. 14, approximately 50% (enumerated below) of the peptides synthesized according to the rules (V) showed 1.5 or higher relative fluorescence intensity values.

| | |
|---|---|
| KGMH, | (SEQ ID NO.:43) |
| KGIR, | (SEQ ID NO.:44) |
| RSVF, | (SEQ ID NO.:45) |
| RGVH, | (SEQ ID NO.:46) |
| KGTR, | (SEQ ID NO.:47) |
| HHYH, | (SEQ ID NO.:48) |
| RIFF, | (SEQ ID NO.:49) |
| KQCR, | (SEQ ID NO.:50) |
| RPVH, | (SEQ ID NO.:51) |
| RFYR, | (SEQ ID NO.:52) |
| KSSY, | (SEQ ID NO.:53) |
| RECY, | (SEQ ID NO.:54) |
| RYNH, | (SEQ ID NO.:55) |
| HLVK, | (SEQ ID NO.:56) |
| RTWR, | (SEQ ID NO.:57) |
| HAQK, | (SEQ ID NO.:58) |
| HFFR, | (SEQ ID NO.:59) |
| KWGK, | (SEQ ID NO.:60) |
| KPDK, | (SEQ ID NO.:61) |
| HMMR, | (SEQ ID NO.:62) |
| HCMY, | (SEQ ID NO.:63) |
| RAMR, | (SEQ ID NO.:64) |
| RVWK, | (SEQ ID NO.:65) |
| KENK, | (SEQ ID NO.:66) |
| REWR, | (SEQ ID NO.:67) |
| HQWY, | (SEQ ID NO.:68) |
| RHDF, | (SEQ ID NO.:69) |
| KAGW, | (SEQ ID NO.:70) |

Among these, KGMH(SEQ ID NO.:43), KGIR(SEQ ID NO.:44), RSVF (SEQ ID NO.:45), RGVH(SEQ ID NO.:46), KGTR(SEQ ID NO.:47), HHYH(SEQ ID NO.:48) have 2.0 or higher relative fluorescence intensity values, thus revealing an extremely high affinity.

Thus, it was demonstrated that the present strategy are extremely effective for designing and synthesizing high-affinity peptides.

### INDUSTRIAL APPLICABILITY

According to the designing method of the present invention, peptides that exhibit a high affinity toward a given target can be efficiently designed. The designing method according to the present invention is applicable for designing peptides that targets proteins such as receptors and antigenic substances. Thus, the designing method according to the present invention is highly universal and applicable to various fields.

The present invention is not limited only to the description of the above embodiments. A variety of modifications which are within the scopes of the following claims and which are achieved easily by a person skilled in the art are included in the present invention. The contents of thesis, published patent application and issued patents cited in the present specification are hereby incorporated by reference in their entirety.

What is claimed is:

1. A method for designing a high-affinity peptide, comprising the steps of (1) to (6) of:
(1) performing an affinity assay using a plurality of peptides having different peptide sequences and a target to obtain affinity data for each of the peptide sequences toward the target;
(2) selecting high-affinity peptide sequences and low-affinity peptide sequences;
(3) digitizing predetermined properties of amino acids for each location from N-terminal or C-terminal to transform each of the selected peptide sequences into numerical data;
(4) performing Fuzzy Neural Network analysis by using the obtained numerical data as input variables to construct a prediction model;
(5) extracting from the constructed prediction model one or two or more rules wherein the amino acids and the properties are related to each other on one or more locations on the sequences, said rules representing the characteristics of high-affinity peptide sequence;
(6) designing a peptide according to the extracted rules.

2. The method according to claim 1, wherein two or more rules are extracted in the step of (6).

3. The designing method according to claim 1, wherein a plurality of peptides having different peptide sequences are designed in the step of (6), further comprising the step of (7) of performing the steps of (1) to (6) using the designed plurality of peptides.

4. A designing method according to claim 3, wherein the step of (7) is repeated two or more times.

5. The designing method according to claim 1, wherein said affinity assay is performed using a peptide chip comprising a plurality of peptides segmented according to each peptide sequence and immobilized on a substrate.

6. The designing method according to claim 1, wherein the plurality of peptides in the step of (1) are equal in length.

7. The designing method according to claim 1, wherein the plurality of peptides in the steps of (1) comprises 3 to 15 amino acids.

8. The designing method according to claim 1, wherein the plurality of peptides in the step of (1) comprises a set of peptides having randomly selected amino acid sequences.

9. The designing method according to claim 1, wherein said target is biopolymer such as cell, protein or peptide, or particulate or base made of metal, semiconductor, inorganic material or synthetic polymer.

10. The designing method according to claim 1, wherein the properties in the step of (3) are one or more properties selected from the group consisting of size, hydrophobicity, charges, isoelectric point, presence or absence of branch, presence or absence of sulfur element, presence or absence of hydroxyl, presence or absence of benzene ring, and presence or absence of heterocycle.

11. The designing method according to claim 10, wherein the properties are two or more properties selected from the above group.

12. The designing method according to claim 1, wherein the properties in the step of (3) are size, hydrophobicity and charges.

13. A method of preparing a high-affinity peptide, comprising preparing a peptide designed by the designing method according to claim 1.

14. Computer-readable storage medium storing a program for performing the following steps to design high-affinity peptides:
digitizing predetermined properties of amino acids for each location from N-terminal or C-terminal to transform into numerical data a plurality of peptide sequences having different affinities toward a given target;
performing Fuzzy Neural Network analysis by using the obtained numerical data as input variables to construct a prediction model; and
extracting from the constructed prediction model one or two or more rules wherein the amino acids and the properties are related to each other on one or more locations on the sequences, said rules representing the characteristics of high-affinity peptide sequence.

15. Computer-readable storage medium storing a program for performing the following steps to design high-affinity peptides:
selecting high-affinity peptide sequences and low-affinity peptide sequences based on affinity data for each of the peptide sequences toward the target, obtained through affinity assay using a plurality of peptides having different sequences and a target;
digitizing predetermined properties of amino acids for each location from N-terminal or C-terminal to transform into numerical data the high-affinity peptide sequences and low-affinity peptide sequences;
performing Fuzzy Neural Network analysis by using the obtained numerical data as input variables to construct a prediction model; and
extracting from the constructed prediction model one or two or more rules wherein the amino acids and the properties are related to each other one or more locations on the sequences, said rules representing the characteristics of high-affinity peptide sequence.

16. Computer-readable storage medium storing a program for performing the following steps to design high-affinity peptides:
digitizing predetermined properties of amino acids for each location from N-terminal or C-terminal to transform into numerical data a plurality of peptide sequences having different affinities toward a given target;
performing Fuzzy Neural Network analysis by using the obtained numerical data as input variables to construct a prediction model;
extracting from the constructed prediction model one or two or more rules wherein the amino acids and the properties are related to each other on one or more locations on the sequences, said rules representing the characteristics of high-affinity peptide sequence; and
designing peptides according to said rule.

17. Computer-readable storage medium storing a program for performing the following steps to design high-affinity peptides:
selecting high-affinity peptide sequences and low-affinity peptide sequences based on affinity data for each of the peptide sequences toward the target, obtained through affinity assay using a plurality of peptides having different sequences and a target;
digitizing predetermined properties of amino acids for each location from N-terminal or C-terminal to transform into numerical data the high-affinity peptide sequences and low-affinity peptide sequences;
performing Fuzzy Neural Network analysis by using the obtained numerical data as input variables to construct a prediction model;
extracting from the constructed prediction model one or two or more rules wherein the amino acids and the properties are related to each other on one or more locations on the sequences, said rules representing the characteristics of high-affinity peptide sequence; and
designing a peptide according to said rules.

18. An apparatus for designing a high-affinity peptide comprising:
means for digitizing predetermined properties of amino acids for each location from N-terminal or C-terminal to transform into numerical data a plurality of peptide sequences having different affinities toward a given target;
means for performing Fuzzy Neural Network analysis by using the obtained numerical data as input variables to construct a prediction model; and
means for extracting from the constructed prediction model one or two or more rules wherein the amino acids and the properties are related to each other on one or more locations on the sequences, said rules representing the characteristics of high-affinity peptide sequence.

19. An apparatus for designing a high-affinity peptide comprising:
means for selecting high-affinity peptide sequences and low-affinity peptide sequences based on affinity data for each of the peptide sequences to ward the target, obtained through affinity assay using a plurality of peptides having different sequences and a target;
means for digitizing predetermined properties of amino acids for each location from N-terminal or C-terminal to transform into numerical data the high-affinity peptide sequences and low-affinity peptide sequences;
means for performing Fuzzy Neural Network analysis by using the obtained numerical data as input variables to construct a prediction model; and
means for extracting from the constructed prediction model one or two or more rules wherein the amino acids and the properties are related to each other on one or more locations on the sequences, said rules representing the characteristics of high-affinity peptide sequence.

20. An apparatus for designing a high-affinity peptide comprising:
means for digitizing predetermined properties of amino acids for each location from N-terminal or C-terminal to transform into numerical data a plurality of peptide sequences having different affinities toward a given target;
means for performing Fuzzy Neural Network analysis by using the obtained numerical data as input variables to construct a prediction model; and
means for extracting from the constructed prediction model one or two or more rules wherein the amino acids and the properties are related to each other on one or more locations on the sequences, said rules representing the characteristics of high-affinity peptide sequence; and
means for designing a peptide according to said rules.

21. An apparatus for designing a high-affinity peptide comprising:
means for selecting high-affinity peptide sequences and low-affinity peptide sequences based on affinity data for each of the peptide sequences to ward the target, obtained through affinity assay using a plurality of peptides having different sequences and a target;
means for digitizing predetermined properties of amino acids for each location from N-terminal or C-terminal to transform into numerical data the high-affinity peptide sequences and low-affinity peptide sequences;
means for performing Fuzzy Neural Network analysis by using the obtained numerical data as input variables to construct a prediction model;
means for extracting from the constructed prediction model one or two or more rules wherein the amino acids and the properties are related to each other on one or more locations on the sequences, said rules representing the characteristics of high-affinity peptide sequence; and
means for designing a peptide according to said rules.

22. A high-affinity peptide comprising an amino acid sequence shown in any one of SEQ ID NOs: 1-70.
